(19) United States
(12) Patent Application Publication
(10) Pub. No.: US 2006/0241347 A1
(43) Pub. Date: Oct. 26, 2006

(54) Systems and methods relating to colposcopic viewing tubes for enhanced viewing and examination

(76) Inventor: Peter Whitehead, (US)

Correspondence Address: GRAYBEAL, JACKSON, HALEY LLP, 155 - 108TH AVENUE NE, SUITE 350, BELLEVUE, WA, 98004-5901, US

(21) Appl. No.: 11/248,604

(22) Filed: Oct. 12, 2005

Related U.S. Application Data
(60) Provisional application No. 60/618,286, filed on Oct. 12, 2004.

(51) Int. Cl. A61B 1/00; A61B 1/06

(52) U.S. Cl. 600/146; 600/129; 600/181; 600/178

(57) ABSTRACT

Colposcopic viewing tubes for use with external colposcopes, otoscopes, velscopes. The colposcopic viewing tube is configured to be placed in front of the viewing lens of the colposcope, is typically opaque for a desired portion, and can create a light-tight connection between the patent and the external colposcope and enhance the accuracy or ease of examination of the cervix and other structures in the vagina. The tubes can, if desired, be flexed, extended, retracted, articulated, etc., while in use and can also be removed from the colposcope and be either sterilizable or disposable. The colposcopic viewing tube can be used with a disposable or sterilizable sleeve. The colposcopic viewing tube can comprise a one or more channels for illumination light, detection, spectroscopy, and imaging. In addition, colposcopic viewing tube can comprise extendable members that can be extended through one or more ports in the colposcopic viewing tube, which extension numbers can be sized and configured to fit within the os or other desired target.

### BACKGROUND

An external colposcope is special kind of microscope used in vaginal examinations. A typical external colposcope looks like a camera, and typically has two eyepieces (is binocular, although colposcopes can also be monocular or have other designs), low-magnification (e.g., 7× to 30×) and a light source. The external colposcope is kept about 3 to 15 inches outside a patient and looks through a speculum that has been placed in the vagina. See, e.g., www.obgyn.net; U.S. Pat. Nos. 6,277,067; 5,791,346; 4,652,103; 4,232, 933; 4,134,637; 3,994,288.

An exemplary prior art external colposcope 2 is shown in FIG. 1 and has a body 4, a pair of oculars or eyepieces 6 and a lens 8. If desired, the eyepieces can be replaced by, or supplemented with, a camera and/or a viewing screen such as a video display. The colposcope 2 is mounted atop a stand 10 that also holds a light and energy source 12. Stand 10 is mounted atop a mobile roller unit 14. In alternative embodiments, the colposcope 12 can be mounted atop a mobile arm that is, in turn, attached to the side of the examination table or other stable element, or the colposcope can be hand-held.

In use, illumination light is emitted from the light emitter 16 towards the patient. Lens 8, which is held outside the patient by about 3 to 15 inches, then picks up returning detection light and transmits it through internal optics (and electronics or other suitable elements, if desired) to eyepieces 6 or other suitable sensor/view screen for review by the doctor. One common goal for the colposcope is to evaluate lesions on the cervix, and thus to detect—and hopefully exclude—the possibility of a malignant cancer.

External colposcopes can suffer from one or more of a variety of difficulties. For example, the ambient room light can hinder the examination, the viewing path for the colposcopes may not conform to the natural shape of the vagina, and certain areas of the cervix and vaginal canal may be difficult to examine, for example because of the shape of the cervix and the enclosed nature of the os (the passage in the cervix leading from the vagina to the inside of the uterus).

Thus, there has gone unmet a need for devices, systems, methods and the like that reduce or remedy one or more of these potential difficulties. The present devices, systems, methods, etc., provide these and other advantages.

### SUMMARY

The present invention provides devices, systems, methods, etc., that enhance the accuracy or ease of examination of the cervix and other structures in the vagina. The devices, etc., comprise a colposcopic viewing tube for use with an external colposcope. The viewing tube creates a light-tight connection between the patent and external colposcope and can, if desired, be flexed, extended, retracted, articulated, etc., while in use. If desired, the colposcope can also be expandable like a speculum, and/or can be attachable to a speculum or fit inside a speculum. The colposcopic viewing tube can also be removed from the colposcope and can be either sterilizable or disposable, and/or the colposcopic viewing tube can be used with a disposable or sterilizable sleeve. The colposcopic viewing tube helps the physician and patient because it reduces hindrances from ambient light (which also reduces the need to lower the ambient lighting in the examination room, thereby increasing the comfort of the patient and facilitating examination of external features at substantially the same time as the internal examination with the colposcope), provides more efficient viewing of curved surfaces such as the outside of the cervix, and/or if desired, helps the ability to examine the os.

These and other aspects, features and embodiments are set forth within this application, including the following Detailed Description and attached drawings. In addition, various references are set forth herein that discuss certain systems, apparatus, methods and other information; all such references are incorporated herein by reference in their entirety and for all their teachings and disclosures, regardless of where the references may appear in this application.

### BRIEF DESCRIPTION OF THE DRAWINGS

FIG. 1 depicts an elevated perspective view of a prior art colposcope.

FIG. 2 depicts a side view of one embodiment of a colposcopic viewing tube according to the present invention.

FIG. 3 depicts a side view of one embodiment of a colposcopic viewing tube according to the present invention wherein the colposcopic viewing tube is removably retained in front of the viewing lens of a colposcope.

FIG. 4 depicts a side view of one embodiment of a distal end of colposcopic viewing tube according to the present invention.

### DETAILED DESCRIPTION

Accurate examination of the cervix and other structures in the vagina is critical for virtually any woman, for example a woman suspected of having cervical cancer or other diseases or conditions involving the cervix or other structures within the vagina. Unfortunately, such structures can be difficult to see, for example because they are inside the woman and because they can have contours that serve to hide different parts of the structures. The present invention provides colposcopic viewing tubes that improve this situation because they reduce interference from ambient light (which also reduces the need to lower the ambient lighting in the examination room, thereby increasing the comfort of the patient and facilitating examination of external features at substantially the same time as the internal examination with the colposcope), more efficient viewing of curved surfaces such as the outside of the cervix, and/or if desired, the ability to examine the os (the passage in the cervix).

The colposcopic viewing tubes can be substantially opaque from at least the colposcope to the patient, and the colposcopic viewing tubes can be articulated (e.g., bent or flexed) to adapt to the internal anatomy of the patient, to approach the sides of the cervix from a more direct angle, or to approach the sides of the vagina from a more direct angle (thereby enhancing the ability to examine the folds of the canal). In addition, the colposcopic viewing tube can comprise one or more extendable members that can be extended through one or more ports (typically in the end but other locations, such as the sides, can also be useful), which extension members can be sized and configured to fit within, and examine, the os or other desired structure.

In some embodiments, the colposcopic viewing tubes comprise a plurality of channels, typically with one or more corresponding ports, that transmit illumination light, detection light emanating from the target, including spectroscopy channels to measure the spectrum emanating from the target and imaging channels to provide an image of the target. If desired, the channels can also provide for suction, biopsy instruments (such as a bioptome or punch biopsy device), marking devices for identifying and tagging desired locations under examination, fluid emission, for example to provide acetic acid or other desirable examination fluids, or therapeutic light such as light from an ablation laser or photodynamic therapy (PDT) laser.

After some discussion about definitions and the like, various embodiments of the present invention will be discussed in conjunction with the Figures, then in more general terms.

All terms used herein, including those specifically discussed below in this section, are used in accordance with their ordinary meanings unless the context or definition clearly indicates otherwise. Also unless indicated otherwise, except within the claims, the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

The scope of the present systems and methods, etc., includes both means plus function and step plus function concepts. However, the terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

Other terms and phrases in this application are defined in accordance with the above definitions, and in other portions of this application.

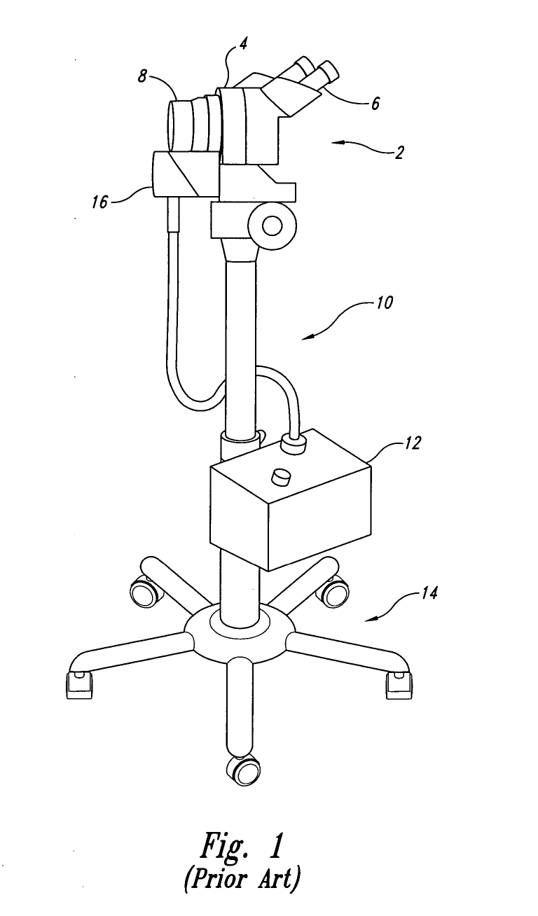

As noted above, FIG. 1 depicts a prior art external colposcope. The term “external colposcope” identifies colposcopes such as that depicted in FIG. 1 (whether handheld, disposed on a rollable stand or other support structure, or otherwise) that are sized and configured to be maintained outside the body of the patient. External colposcope does not refer to endoscopes sized and configured to be used within the body.

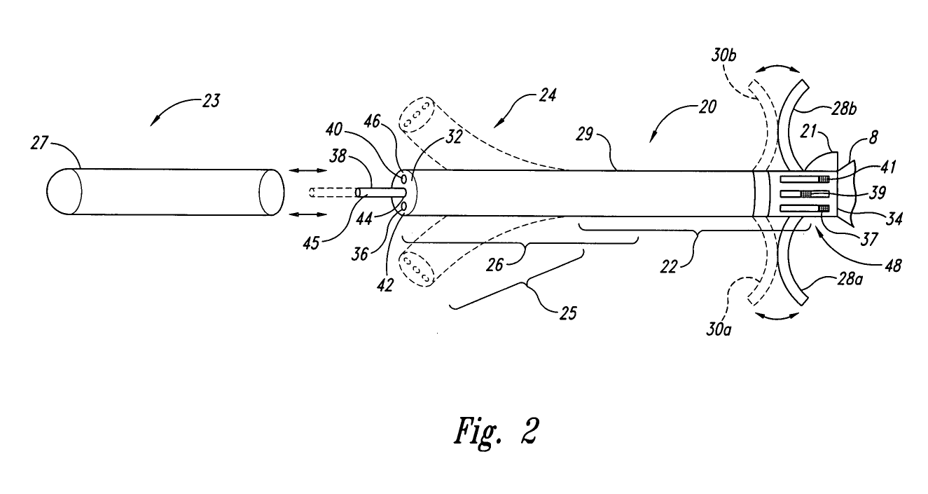

FIG. 2 depicts one embodiment of a colposcopic viewing tube 20 according to the present invention. As depicted, the colposcopic viewing tube comprises a body 29 that is substantially circular in cross section. Other cross sectional shapes can also be used if desired. Colposcopic viewing tube 20 can be made of plastic, metal, or any other medically acceptable material.

Colposcopic viewing tube 20 as depicted is removable from a viewing lens 8, which indicates that the colposcopic viewing tube can be removably retained in front of the viewing lens 8 so that the colposcope can be used both with and without the colposcopic viewing tube 20. In other embodiments, the colposcopic viewing tube can be permanently attached. In such embodiments, or for ease or otherwise as desired, the colposcopic viewing tube system can also comprise a removable, optionally disposable sleeve 23 (not drawn to scale), so that the colposcopic viewing tube can be used repeatedly without cleaning and/or sterilization. Sleeve 23 is itself an advancement forming a part of the present invention, and can be of varied or variable opacity, if desired. For example, sleeve 23 can comprise a distal transparent portion 27. In other embodiments of colposcopic viewing tube 20, the body 29, or other desired parts can be detachable or disposable.

Colposcopic viewing tube 20 comprises a proximal end 48 that ends in a proximal tip 34 and a distal end 24 that ends in a distal tip 32, and is sized to be inserted into the vagina. Proximal tip 34 is typically sized to abut or be maintained in close proximity to the front of viewing lens 8. The proximal end is the end of the colposcopic viewing tube that is maintained outside the body, and typically comprises one or more ports such as openings, passages, channels, etc., that convey information between the target and the external colposcope or other proximally-located devices such as a spectrometer (if the spectrometer is not a part of the colposcope itself), and analyzing computers or other programmed controllers. The proximal end typically comprises one or more handles 28a, 28b, knobs 37, 39, 41, or other extension control members that allow a user to manipulate articulatable portion 25 of the colposcopic viewing tube 20 or the devices located in the distal end 24 of the colposcopic viewing tube 20. The distal end 24 of the colposcopic viewing tube 20 includes distal tip 32, which is the most distal surface or opening of the viewing tube, whereas the portion of the colposcopic viewing tube 20 including and adjacent to the distal tip 24 of the colposcopic viewing tube 20 is the distal end 24. The distal tip/end (and certain other portions, if desired) can be open or enclosed.

Typically, external colposcopes are maintained about 3-15 inches (about 10-40 cm) outside of the patient, so such insertion typically involves inserting only an insertable portion 26 of colposcopic viewing tube 20. Accordingly, colposcopic viewing tube 20 typically also comprises a substantially rigid portion 22, which typically extends from the viewing lens 8 to the articulatable portion 25. As depicted, insertable portion 26 and substantially rigid portion 22 overlap; such portions can also abut or be separated. If desired, the substantially rigid portion can be bendable or adjustable, for example to enhance ease of viewing for the physician when examining a patient located unusually high or low on the examination table.

If colposcopic viewing tube 20 comprises a significant deviation in its course or light paths, or for other reasons such as enhanced clarity or magnification, then suitable optical elements, such as lenses, mirrors, lights guides, etc., can be incorporated into or with colposcopic viewing tube 20 to direct or modify any images or other examination data to the eyepieces, the colposcope or other desired recipient.

Substantially articulatable portion 25 can be articulated in advance by bending the device prior to insertion, or as in the embodiment shown, can be articulated while in the examination position via the manipulation of one or more articulation control members 28a, 28b that are operably connected (e.g., mechanically, electrically, etc.) to the substantially articulatable portion 25. In the embodiment depicted, the articulation control members 28a, 28b are handles that can be twisted from a first position (shown in hard line) to other positions, such as alternative positions 30a, 30b shown in dotted line. Also disposed at proximal end 48 are a plurality of extension control members, typically positioned on the colposcopic viewing tube 20 to be maintained outside of the patient. As depicted, the extension control members are knobs 37, 39, 41, that can be pushed back and forth to extend and retract extendable elements 36, 38 and 40.

In FIG. 2, extendable element 36 is an illumination light emitter that conveys illumination light from an integral light source 21 attached directly to the proximal end of the colposcopic viewing tube 20; an integral light source can also be maintained inside or attached at other locations along the colposcopic viewing tube 20. In other words, as depicted the illumination light channel 43 is configured to operably transmit illumination light to the target from the illumination light source maintained at the proximal end of the viewing tube, the illumination light channel 43. As depicted, the illumination light channel 43 is fiber optic light guide, although any other desired light guide, such as a fiber optic bundle, liquid light guide, etc., could be used if desired. Imaging channel 38 occupies a central position and is partially extended.

As with the other channels, imaging channel 38 is operably connected to extension control member 39 such that moving extension control member 39 back and forth extends and retracts imaging channel 38 as an extendable element. If desired, in certain embodiments, multiple functions can be maintained in a single channel. For example, imaging channel 38 could also be a light emitting channel and a spectroscopy channel. As depicted, imaging channel 38 is a fiber optic light guide bundle wherein a plurality of small fiber optics are bundled together to transmit the image to the proximal end. Imaging channel 38 passes through imaging channel port 44; corresponding ports 42, 46 are set in distal tip 32 for illumination light emitter 36 and spectroscopy channel 40. As depicted in FIG. 2, extendable element 38 is sized and configured to fit in an os. Also as depicted in FIG. 2, the port(s) are in the distal tip 32 of the colposcopic viewing tube 20; ports can also, or alternatively, be located in the sides of the colposcopic viewing tube 20.

Distal tip 32 of colposcopic viewing tube 20 is typically substantially flat to enhance examination. The tip need not be precisely flat. For example, a partial curve or curves, as shown in FIG. 4, which may be in a spherical, toroidal, or other desired shape, may also be employed yet can be substantially flat, such that when the distal tip is contacted on a cervix or other structure under examination, the distal tip substantially flattens a corresponding portion of the cervix or other target for examination. The substantially flat distal tip 32 can be configured at a substantially right angle to the axis of the colposcopic viewing tube 20. The target need not be completely flattened unless desired. Flattening the target can be advantageous because it can reduce the curvature of the cervix or the folds along the interior of the vagina, thereby enhancing examination. In the embodiment depicted in FIG. 2, distal tip 32 is enclosed; in other embodiments, the distal tip of the colposcopic viewing tube 20 can be open.

The portion of the colposcopic viewing tube extending between the colposcope and the patient can be substantially light proof (if desired, all of the viewing tube but any desired light ports can be substantially light proof). Such a configuration is advantageous, for example, because it reduces the interference of ambient light in the examination process. Thus, a substantially light proof connection can be established between the colposcope and the patient.

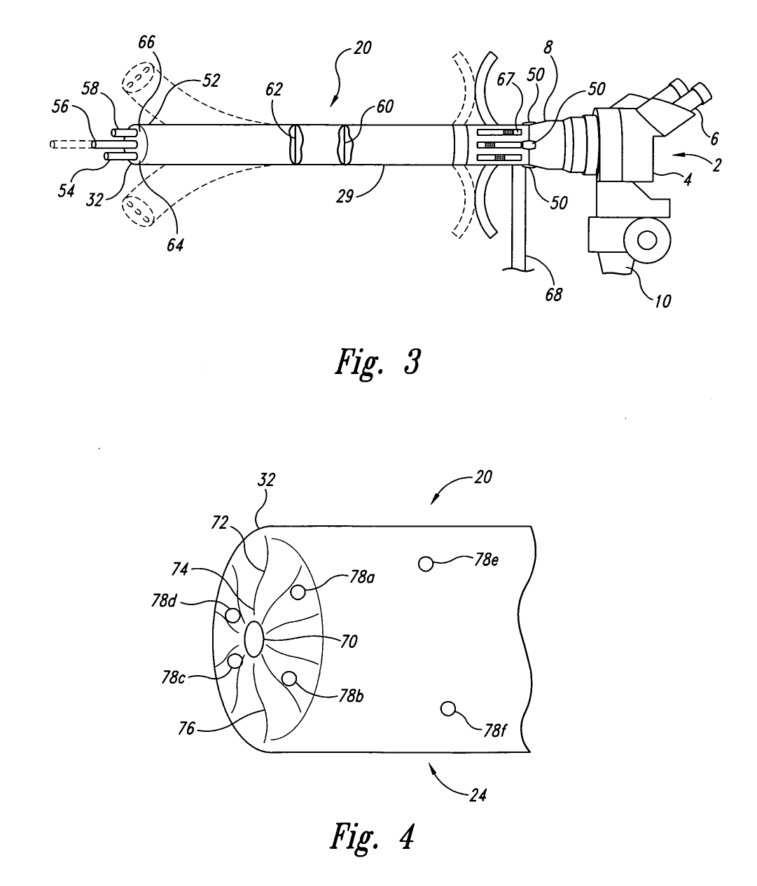

The various channels can be separate channels or can be a single channel. For example, it can be advantageous for all of the channels to be maintained, at least at the distal end, in a single channel that is sized to extend and retract and possibly to fit inside an os. This can be particularly advantageous where the channels all carry light because multiple different structures need not be included in the extendible element. Such can also be the arrangement for other channels, for example channels where a bioptome is combined with a light channel in a single device. See, e.g., U.S. Pat. No. 6,201,989. Conversely, when a function such as biopsy procurement is provided by one channel (for example via provision of a biopsy device such as bioptome 58 in FIG. 3, discussed further below) then it can be advantageous to maintain both the illumination light and the imaging light in other channel(s) so that the physician can see the area from which the biopsy is being taken. Thus, the various channels can be a single channel or can be separate channels (as used herein, separate channels indicates different channels that are all a part of the viewing tube, not an additional viewing tube).

FIG. 3 depicts an external colposcope 2 comprising a viewing lens 8 and a colposcopic viewing tube 20 maintained in front of the viewing lens, and sized to be inserted into a vagina, and having a substantially rigid proximal portion and an articulatable distal end. The colposcopic viewing tube 20 can alternatively or also comprise an illumination light emitter configured to emit illumination light from the distal end, for example from a distal light emitter 64 or an external light source that provides light via a light guide 68, and also can comprise a spectroscopy channel and an imaging channel.

Distal end 32 is substantially transparent and therefore provides an illumination and viewing port 52. Moreover, in this embodiment distal end 32 is not substantially flat but is instead domed; other non-substantially flat shapes are also possible. Suction channel 54, liquid emitter 56 (which emitter could also be configured to emit other compositions or substances such as gasses, powders, etc.), bioptome 58, and therapeutic light channel 66 extend and retract through distal tip 32. As depicted, colposcopic viewing tube 20 is removably retained by a plurality of clips 50. Colposcopic viewing tube 20 can be retained in front of viewing lens 8 by any other suitable devices if desired, such as straps, hook-and-loop connectors such as Velcro™, threadings such that colposcopic viewing tube 20 screws onto the case of viewing lens 8, etc. In addition, the actual attachment points, if any, need not be direct to the viewing lens 8 or its immediate case; colposcopic viewing tube 20 could be attached to body 4, stand 10, light source 12, or even to a device not connected to the colposcope or its support structure, but movable such that it can place and maintain colposcopic viewing tube 20 in front of (i.e., abutting or proximate to) viewing lens 8.

In FIG. 3, an illumination wavelength selective element 62 and a detection wavelength selective element 60 are maintained within colposcopic viewing tube 20 in the detection and illumination light path (which as depicted is a single channel). The wavelength selective elements can be any desired optical elements suitable for a desired purpose, such as filters, beam splitters, spectrum formers, etc. The wavelength selective elements are located and configured to selectively substantially inhibit at least one non-desired wavelength from the light from being transmitted out of the distal end of the viewing tube to a target or out of the proximal end to the viewing lens 8 or other desired receiver. In one embodiment, and as depicted, the illumination wavelength selective element 62 is a short pass element such as a short pass filter configured to substantially transmit blue light. Such a configuration is advantageous because it provides a safe yet fluorescence-inducing light. The detection wavelength selective element 60 can be a long pass element configured to substantially block blue (and UV) light and to substantially transmit wavelengths of light longer than the blue light. For example, such a long pass element can be a long pass filter configured to substantially block fluorescence-inducing illumination light that has been reflected from the target and to substantially transmit fluorescence light emitted or emanated from the target to the viewer. Other desired wavelength selective elements and combinations can also be used, e.g., U.S. Pat. No. 6,110,106.

In FIG. 3, both a proximal white light source and an auxiliary distal light source 64 are provided in the system. The proximal light source can be the light source 12 initially provided with the external colposcope itself, while distal light source 64 can be maintained at the distal end 24 of colposcopic viewing tube 20. The light source can also be integral to the colposcopic viewing tube 20, such as integral light source 21 depicted in FIG. 2. Such provision of multiple light sources can be advantageous for the provision of different light for different examination purposes. For example, distal light source 64 and the other light sources can comprise, or exclude, a blue LED, a UV light source, an IR light source or a white light source.

In some embodiments, such as shown in FIG. 2, colposcopic viewing tube 20 comprises an illumination light emitter 36 that is configured to emit light from distal end 24 to a target. The colposcopic viewing tube 20 can further comprise at least one of a spectroscopy channel configured to operably transmit (i.e., transmit in usable format) detection light from the target to a measurement device such as a spectrometer or spectrophotometer, or transmit the light to a lead going to a measurement device maintained outside the patient. The colposcopic viewing tube 20 can be an imaging channel that is configured to operably transmit an image of the target from the target to the viewing lens. The illumination light emitter can be either an illumination light channel 43 configured to operably transmit illumination light to the target from illumination light source 12 maintained proximally to colposcopic viewing tube 20 as depicted in FIG. 3, or the illumination light emitter can be an illumination light source such as illuminating distal light source 64 also depicted in FIG. 3. Such embodiments can be useful in colposcopic viewing tubes even if the colposcopic viewing tubes do not have an articulatable or substantially flexible end and/or do not comprise a substantially light proof, or opaque, sleeve containing the various channels.

FIG. 4 depicts one embodiment of a distal tip 32 of a distal end 24 of colposcopic viewing tube 20. The distal tip 32, which comprises a central port 70 and additional ports 78a-d, is shaped to complement a cervix of patient, as indicated by contour lines 72. Accordingly, the distal tip 32 has a substantially circular depression 76 and a bulge 74. Such a shape can enhance the ability of the colposcopic viewing tube 20 to flatten the cervix during examination or other use, while also assisting in the placement of central port 70 (and any elements corresponding thereto, such as an illumination light channel, an imaging channel, a spectroscopy channel, a biopsy channel, etc.) at the os, as well as placing other ports 78a-d at desired positions along the cervix. Other shapes can also be provided that enhance examination of the cervix, the walls of the vagina, etc., or otherwise assist the examiner. For example, as also shown in FIG. 4, the distal end 24 also comprises side-viewing ports 78e and 78f. Such can be useful for viewing the walls of the vagina, etc.

Turning to a more general discussion of the invention, the present invention comprises colposcopic viewing tubes sized and configured specifically for external colposcopes, to provide enhanced examination of the vagina and structures located therein, such as the cervix and os. In certain embodiments, the colposcopic viewing tube provides a substantially light proof connection between the external colposcope and the target within the patient, provides one or more specialized channels operably connected between the distal end of the colposcopic viewing tube and proximally maintained devices such as the colposcope, a spectrometer, video devices, digital imaging equipment and computers. The various channels typically comprise an illumination light emitter (either a light source maintained at the distal end of the colposcopic viewing tube and/or a illumination light channel to convey light from a proximal light source to the target), an imaging channel and a spectroscopy channel. If desired, a variety of other channels can be used with, or in place of, one or more of such channels including, for example, a vacuum channel, a fluid (or other substance) channel, a vacuum channel, a biopsy channel, and a therapeutic light channel for a laser or other coherent light source, for example an ablation laser or a photodynamic therapy light source.

If desired, the colposcopic viewing tube can be provided with one or more filters, lenses, mirrors, beam splitters, or other optical elements such that certain desired light is emitted for illumination purposes and only certain desired light is gathered or transmitted for detection purposes. This can be advantageous, for example, when using a short wavelength light such as blue light to illuminate the target and induce fluorescence such that blocking wavelengths of light other than blue light is helpful for the fluorescence induction, and blocking the blue light (or other fluorescence inducing light) reflecting off the target is helpful for observation and spectral analysis.

The colposcopic viewing tube is typically at least semi-rigid between the external colposcope and the patient and further can have an articulatable distal end sized and configured to adapt to the physiology of a particular woman and to enhance examination of tissues having complex geometries such as folds within the vagina and the donut-shaped cervix. The articulation of the distal end can be either performed annually prior to insertion or can be performed via one or more articulation control members maintained outside the patient.

Turning to some additional discussion of some methods aspects of the present invention, the methods include preparing an external colposcope comprising: a) providing an external colposcope comprising a viewing lens; and b) placing a colposcopic viewing tube in operable connection in front of the viewing lens, the colposcopic viewing tube sized to be inserted into a vagina and comprising a substantially rigid proximal portion and an articulatable distal end. The placing further can comprise attaching the colposcopic viewing tube to the viewing lens.

The methods further can comprise removing the colposcopic viewing tube from the viewing lens, and articulating the distal end by manipulating at least one articulation control member operably linked to the distal end. The colposcopic viewing tube can be substantially opaque between at least a proximal end and an insertable portion, to provide a substantially light proof connection between the external colposcope and a patient. Thus, the methods can comprise obtaining examination information from a patient with reduced, or even without significant, interference from ambient or other undesired light. Such methods can also comprise obtaining such information without dimming the room lights. The methods can also comprise removably placing a disposable sleeve on the colposcopic viewing tube, the sleeve comprising a distal transparent portion.

The colposcopic viewing tube can also have at least one illumination wavelength selective element located and configured to selectively substantially inhibit at least one non-desired wavelength of illumination light from being transmitted out of the distal end of the colposcopic viewing tube, and the methods can comprise transmitting a beam of illumination light through the colposcopic viewing tube and selectively inhibiting the at least one non-desired wavelength of illumination light from being transmitted out of the distal end of the colposcopic viewing tube. The colposcopic viewing tube can have at least one detection wavelength selective element located and configured to selectively substantially inhibit at least one non-desired wavelength of detection light from being transmitted out of the proximal end of the colposcopic viewing tube to at least one of the viewing lens and a spectral measurement device, and the methods further can comprise transmitting a beam of detection light through the colposcopic viewing tube and selectively inhibiting the at least one non-desired wavelength of detection light from being transmitted out of the proximal end of the colposcopic viewing tube.

The colposcopic viewing tube further can comprise an illumination light emitter configured to emit illumination light from a distal end of the colposcopic viewing tube to a target, and the methods further can comprise emitting light from the distal end of the colposcopic viewing tube, and the tube can comprise at least one of a spectroscopy channel and an imaging channel, and the methods can comprise at least one of transmitting detection light through the spectroscopy channel and transmitting an image through the imaging channel. If desired, at least one of the illumination light emitter, the spectroscopy channel and the imaging channel can be configured to controllably extend and retract from the distal end of the colposcopic viewing tube, to provide at least one extendable element, and the methods can further comprise extending and retracting the extendable element. The extendable element can be sized and configured to fit inside an os.

In other aspects, the present invention comprises methods of preparing an external colposcope for an examination. Such methods can comprise a) providing an external colposcope comprising a viewing lens and b) placing a colposcopic viewing tube in operable connection in front of the viewing lens, for example by attaching the tube to the lens, wherein the colposcopic viewing tube comprises an illumination light emitter at a distal end, a separate spectroscopy channel and a separate imaging channel configured to operably transmit an image of the target from the target to the viewing lens. If desired, in other embodiments all or some of the channels can be carried in a single structure, such as a liquid light guide, a fiber optic, a fiber optic bundle, or any other desired and suitable light-carrying device. The colposcopic viewing tube can also have at least one of a biopsy channel, a vacuum channel, a therapeutic light channel, a liquid emission channel or other desired channel.

As discussed elsewhere herein, the colposcopic viewing tube can also comprise a substantially rigid proximal portion and an articulatable distal end, can be substantially opaque between at least a proximal end and an insertable portion, and can comprise one or more wavelength selective elements. The methods can also comprise removably placing a disposable sleeve comprising a distal transparent portion on the colposcopic viewing tube, and one or more of the channels can be configured to controllably extend and retract from the distal end of the colposcopic viewing tube, such that the methods comprise extending and retracting the extendable element, for example into and out of an os, or into and out of a fold in the vaginal wall.

In still other aspects, the methods comprise using an external colposcope and/or colposcopic viewing tube as discussed herein, typically for examining or otherwise caring for a patient. The methods can be as follows: provide the external colposcope and colposcopic viewing tube, insert the colposcopic viewing tube into a vagina of the patient then obtain information about a target inside the vagina through the colposcopic viewing tube. The methods herein include changing the order of various elements of the methods where practicable. For example, such methods include inserting the viewing the tube then placing it in front of the colposcope. The methods can further comprise articulating the distal end inside the patient, for example by manipulating at least one articulation control member, such a button, handle, or electronic control. As also discussed elsewhere, the colposcopic viewing tube can be substantially opaque in desired portions, and can comprise a substantially flat distal tip. In such embodiments, the methods can further comprise substantially flattening a target structure, such as an os or vaginal fold, inside the vagina prior to (i.e., as a part of) obtaining the information, and/or extending an extendable member from the colposcopic viewing tube. The methods typically also comprise removing the colposcopic viewing tube from the vagina, and can comprise removing the colposcopic viewing tube from in front of the viewing lens.

The methods can also include emitting illumination light from an illumination light emitter located at the distal end, and collecting information comprising at least an image and a spectral analysis of the target; the information can, if desired, include physical information such as a biopsy, and the methods can also include performing actions on the target, such as emitting fluids such as water, saline solution or acetic acid, injecting drugs or other therapeutically active substances, for example through a needle (which can be extendable), performing ablation or photodynamic therapy treatments (or other light-based treatments), vacuuming up material at the target site, or otherwise treating, diagnosing, examining and caring for the patient.

From the foregoing, it will be appreciated that, although specific embodiments have been discussed herein for purposes of illustration, various modifications may be made without deviating from the spirit and scope of the disclosure. Accordingly, the systems and methods, etc., include such modifications as well as all permutations and combinations of the subject matter set forth herein and can be not limited except as by the appended claims.

1. A colposcopic viewing tube for an external colposcope, the colposcopic viewing tube configured to be removably retained in front of a viewing lens of the colposcope and sized to be inserted into a vagina, wherein the colposcopic viewing tube comprises a substantially rigid proximal portion and an articulatable distal end.

2. The colposcopic viewing tube of claim 1 wherein the colposcopic viewing tube further comprises at least one articulation control member at a proximal end, the articulation control member operably linked to the distal end to controllably articulate the distal end.

3. The colposcopic viewing tube of claim 1 wherein the colposcopic viewing tube is substantially opaque between at least a proximal end and an insertable portion, to provide a substantially light proof connection between the colposcope and a patient.

4. The colposcopic viewing tube of claim 1 wherein the colposcopic viewing tube comprises a substantially flat distal tip.

5. The colposcopic viewing tube of claim 4 wherein the substantially flat distal tip is at a substantially right angle to the axis of the colposcopic viewing tube.

6. The colposcopic viewing tube of claim 1 wherein the colposcopic viewing tube comprises a substantially domed distal tip.

7. The colposcopic viewing tube of claim 1 wherein the distal tip is shaped such that, upon contacting a cervix of a patient, the distal tip substantially flattens a corresponding portion of the cervix that is sized for examination.

8. The colposcopic viewing tube of claim 1 wherein the colposcopic viewing tube further comprises at least one illumination wavelength selective element located and configured to selectively substantially inhibit at least one non-desired wavelength of illumination light from being transmitted out of the distal end of the colposcopic viewing tube to a target.

9. The colposcopic viewing tube of claim 8 wherein the illumination wavelength selective element comprises a short pass element configured to substantially transmit blue light.

10. The colposcopic viewing tube of claim 1 or 8 wherein the colposcopic viewing tube further comprises at least one detection wavelength selective element located and configured to selectively substantially inhibit at least one non-desired wavelength of detection light from being transmitted out of the proximal end of the colposcopic viewing tube to at least one of the viewing lens and a spectral measurement device.

11. The colposcopic viewing tube of claim 10 wherein the detection wavelength selective element comprises a long pass element configured to substantially block blue and UV light and to substantially transmit wavelengths of light longer than blue light.

12. The colposcopic viewing tube of claim 10 wherein the detection wavelength selective element comprises a long pass element configured to substantially block fluorescence-inducing illumination light and to substantially transmit fluorescent light.

13. The colposcopic viewing tube of claim 1 wherein the colposcopic viewing tube further comprises an illumination light emitter configured to emit illumination light from a distal end of the colposcopic viewing tube to a target, and at least one of a spectroscopy channel configured to operably transmit detection light from the target to a spectral measurement device maintained outside a patient and an imaging channel configured to operably transmit an image of the target from the target to the viewing lens.

14. The colposcopic viewing tube of claim 13 wherein the colposcopic viewing tube further comprises an illumination light channel configured to operably transmit illumination light to the target from an illumination light source maintained proximally to the colposcopic viewing tube.

15. The colposcopic viewing tube of claim 14 wherein the illumination light comprises at least one of a metal halide light source, a halogen light source, a laser arc light source, or a light source from a spectrometer.

16. The colposcopic viewing tube of claim 14 wherein the illumination light channel comprises a fiber optic light guide.

17. The colposcopic viewing tube of claim 13 wherein the illumination light emitter comprises a distal light source disposed at the distal end of the colposcopic viewing tube.

18. The colposcopic viewing tube of claim 17 wherein the distal light source comprises a blue LED.

19. The colposcopic viewing tube of claim 13 wherein the spectroscopy channel and the imaging channel are a single channel.

20. The colposcopic viewing tube of claim 14 wherein the illumination light channel, the spectroscopy channel and the imaging channel are a single channel.

21. The colposcopic viewing tube of claim 14 wherein the illumination light channel, the spectroscopy channel and the imaging channel are each separate channels.

22. The colposcopic viewing tube of claim 13 or 20 wherein the colposcopic viewing tube further comprises at least one illumination wavelength selective element located in an illumination light path of the colposcopic viewing tube and configured to selectively substantially inhibit at least one non-desired wavelength of illumination light from being transmitted out of the distal end of the colposcopic viewing tube to a target.

23. The colposcopic viewing tube of claim 13 or 20 wherein the colposcopic viewing tube further comprises at least one detection wavelength selective element located in a detection light path of the colposcopic viewing tube and configured to selectively substantially inhibit at least one non-desired wavelength of detection light from being transmitted out of the proximal end of the colposcopic viewing tube to at least one of the viewing lens and the spectral measurement device.

24. The colposcopic viewing tube of claim 13 wherein at least one of the illumination light emitter, the spectroscopy channel and the imaging channel is configured to controllably extend and retract from the distal end of the colposcopic viewing tube, to provide at least one extendable element.

25. The colposcopic viewing tube of claim 24 wherein the illumination light emitter, the spectroscopy channel and the imaging channel are separate and each controllably extends and retracts from the distal tip of the colposcopic viewing tube, to provide a plurality of extendable elements.

26. The colposcopic viewing tube of claim 25 wherein the distal end of the colposcopic viewing tube is enclosed and the distal end further comprises a plurality of ports at least equal to the plurality of extendable elements, the ports sized to permit passage of the extendable elements.

27. The colposcopic viewing tube of claim 25 or 26 wherein the extendable element is sized and configured to fit inside an os.

28. The colposcopic viewing tube of claim 27 wherein the extendable element comprises at least one side viewing port.

29. The colposcopic viewing tube of claim 14 or 16 wherein the colposcopic viewing tube further comprises at least one extension control member positioned on the colposcopic viewing tube to be maintained outside of the patient, the extension control member operably attached to a corresponding extendable element to control the amount of extension and retraction of the extendable element.

30. The colposcopic viewing tube of claim 13 wherein the spectroscopy channel comprises a light guide.

31. The colposcopic viewing tube of claim 13 wherein the illumination light channel comprises a light guide.

32. The colposcopic viewing tube of claim 13 wherein the colposcopic viewing tube further comprises at least one of a biopsy channel, a vacuum channel, a therapeutic light channel, and a liquid emission channel.

33. A removable colposcopic viewing tube for an external colposcope, the colposcopic viewing tube configured to be removably retained in front of a viewing lens of the colposcope and sized to be inserted into a vagina, wherein the colposcopic viewing tube comprises an illumination light emitter configured to emit illumination light from a distal end of the colposcopic viewing tube to a target, a separate spectroscopy channel configured to operably transmit detection light from the target to a spectral measurement device maintained outside a patient and a separate imaging channel configured to operably transmit an image of the target from the target to the viewing lens.

34-51. (canceled)

52. An external colposcope comprising a viewing lens, wherein the improvement comprises a colposcopic viewing tube retained in operable connection in front of the viewing lens, the colposcopic viewing tube sized to be inserted into a vagina, wherein the colposcopic viewing tube comprises a substantially rigid proximal portion and an articulatable distal end.

53-65. (canceled)

66. An external colposcope comprising a viewing lens, wherein the improvement comprises a colposcopic viewing tube retained in operable connection in front of the viewing lens, the colposcopic viewing tube sized to be inserted into a vagina, wherein the colposcopic viewing tube comprises an illumination light emitter configured to emit illumination light from a distal end of the colposcopic viewing tube to a target, a separate spectroscopy channel configured to operably transmit detection light from the target to a spectral measurement device maintained outside a patient and a separate imaging channel configured to operably transmit an image of the target from the target to the viewing lens.

66-73. (canceled)

74. A method of preparing an external colposcope comprising:
a) providing an external colposcope comprising a viewing lens; and
b) placing a colposcopic viewing tube in operable connection in front of the viewing lens, the colposcopic viewing tube sized to be inserted into a vagina and comprising a substantially rigid proximal portion and an articulatable distal end.

75-86. (canceled)

87. A method of preparing an external colposcope for an examination comprising:
a) providing an external colposcope comprising a viewing lens; and
b) placing a colposcopic viewing tube in operable connection in front of the viewing lens, the colposcopic viewing tube sized to be inserted into a vagina, wherein the colposcopic viewing tube comprises an illumination light emitter configured to emit illumination light from a distal end of the colposcopic viewing tube to a target, a separate spectroscopy channel configured to operably transmit detection light from the target to a spectral measurement device maintained outside a patient and a separate imaging channel configured to operably transmit an image of the target from the target to the viewing lens.

88-96. (canceled)

97. A method of using an external colposcope comprising:
a) providing an external colposcope comprising a viewing lens and a colposcopic viewing tube in operable connection in front of the viewing lens, the colposcopic viewing tube sized to be inserted into a vagina and comprising a substantially rigid proximal portion and an articulatable distal end;
b) inserting the colposcopic viewing tube into a vagina of a patient; and
c) obtaining information about a target inside the vagina through the colposcopic viewing tube.

98-105. (canceled)

106. A method of using an external colposcope comprising:
a) providing an external colposcope comprising a viewing lens and a colposcopic viewing tube in operable connection in front of the viewing lens, the colposcopic viewing tube sized to be inserted into a vagina, wherein the colposcopic viewing tube comprises an illumination light emitter configured to emit illumination light from a distal end of the colposcopic viewing tube to a target, a separate spectroscopy channel configured to operably transmit detection light from the target to a spectral measurement device maintained outside a patient and a separate imaging channel configured to operably transmit an image of the target from the target to the viewing lens;
b) inserting the colposcopic viewing tube into a vagina of a patient and emitting light from the illumination light emitter; and
c) obtaining information about a target inside the vagina through at least one of the spectroscopy channel and the imaging channel.

107-114. (canceled)

115. A kit containing a colposcopic viewing tube according to claim 1 or 33 and a label identifying the colposcopic viewing tube, wherein language on the label has been approved by a governmental health-related regulatory agency.

116. The kit of claim 115 wherein the governmental regulatory agency is the FDA.

117-118. (canceled)
